(19) United States
(12) Patent Application Publication
(10) Pub. No.: US 2025/0235481 A1
(43) Pub. Date: Jul. 24, 2025

(54) GENETICALLY ENGINEERED MESENCHYMAL STEM CELLS AND USES THEREOF

(71) Applicant: CHINA MEDICAL UNIVERSITY, Taichung (TW)

(72) Inventors: Long-Bin JENG, Taichung City (TW); Mien-Chie HUNG, Taichung City (TW); Woei-Cherng SHYU, Taichung (TW); Chih-Hao WANG, Taichung City (TW)

(21) Appl. No.: 19/032,393

(22) Filed: Jan. 20, 2025

Related U.S. Application Data
(60) Provisional application No. 63/622,657, filed on Jan. 19, 2024.

(51) Int. Cl. A61K 35/28; A61P 1/16; C07K 14/47; C07K 14/705; C12N 5/0775; C12N 9/22; C12N 15/11; C12N 15/85

(52) U.S. Cl. CPC A61K 35/28; A61P 1/16; C07K 14/4702; C07K 14/70567; C12N 5/0665; C12N 9/22; C12N 15/111; C12N 15/85; C12N 2310/20; C12N 2510/00

(57) ABSTRACT

Disclosed herein is a genetically engineered mesenchymal stem cells (MSC) comprising genetic alterations that increase gene expressions for adipogenic differentiation, mitochondrial biogenesis and a brown adipocyte marker. Also disclosed herein is a pharmaceutical composition and use of the pharmaceutical composition for preventing, ameliorating and/or treating a metabolic disorder.

### PRIORITY INFORMATION

This application claims benefit of and priority to U.S. Provisional Patent Application No. 63/622,657, filed Jan. 19, 2024, the contents of which is incorporated by reference in its entirety.

### FIELD OF THE INVENTION

This disclosure generally concerns a field of engineered stem cells and its applications. Particularly, the engineered stem cells can be used to treat or ameliorate a metabolic disorder.

### BACKGROUND OF THE INVENTION

The global prevalence of obesity and related metabolic disorders is increasing, leading to elevated rates of morbidity and mortality. While obesity-associated hepatic steatosis is often benign in its clinical presentation, it can quickly progress to metabolic dysfunction-associated steatotic liver disease (MASLD), a serious metabolic liver condition characterized by persistent inflammation, liver injury, and fibrosis. MASLD significantly heightens the risk of end-stage liver diseases, including cirrhosis and hepatocellular carcinoma (HCC). Despite numerous efforts to develop pharmacological therapies targeting MASLD, many have failed in clinical trials.

### SUMMARY OF THE INVENTION

The following presents a simplified summary of the disclosure in order to provide a basic understanding to the reader. This summary is not an extensive overview of the disclosure and it does not identify key/critical elements of the present invention or delineate the scope of the present invention. Its sole purpose is to present some concepts disclosed herein in a simplified form as a prelude to the more detailed description that is presented later.

As embodied and broadly described herein, one aspect of the disclosure is directed to a genetically engineered mesenchymal stem cells (MSC). In some embodiments of the disclosure, the MSC comprises genetic alterations that increase gene expressions for adipogenic differentiation, mitochondrial biogenesis and a brown adipocyte marker. In some embodiments of the disclosure, the MSC comprises exogenous genes of comprising adipogenic differentiation, mitochondrial biogenesis and a brown adipocyte marker, wherein the gene expressions of adipogenic differentiation, mitochondrial biogenesis and a brown adipocyte marker are increased.

In some embodiments of the disclosure, the genetic alterations increase gene expression of peroxisome proliferator-activated receptor gamma (PPARγ).

In some embodiments of the disclosure, the genetic alterations increase gene expression of peroxisome proliferator-activated receptor-γ coactivator 1-α (PGC1α).

In some embodiments of the disclosure, the genetic alterations increase gene expression of mitochondrial uncoupling protein 1 (UCP1).

In one embodiment, the present disclosure provides a genetically engineered mesenchymal stem cell (MSC) comprising one, two or three exogenous genes selected from peroxisome proliferator-activated receptor gamma (PPARγ), peroxisome proliferator-activated receptor-γ coactivator 1-α (PGC1α) and mitochondrial uncoupling protein 1 (UCP1), wherein the gene expressions of PPARγ, PGC1α and UCP1 are increased.

In some embodiments, the genetically engineered MSC comprises an exogenous promoter, an exogenous nucleic acid region encoding PPARγ, an exogenous nucleic acid region encoding PGC1α and an exogenous nucleic acid region encoding UCP1.

In one embodiment, the present disclosure provides a method for preparing a population of genetically engineered MSCs, comprising preparing a vector comprising all-in-one CRISPR-based multigene activation plasmid, wherein the plasmid is prepared by providing gRNA cloning vectors comprising recombination sites and PPARγ, PGC1a and UCP1, respectively, and mixing the gRNA cloning vectors with a transposon backbone vector containing CRISPR/nuclease to generate all-in-one CRISPR-based multigene activation plasmid; transfecting MSCs with the vectors to generate genetically engineered MSCs, culturing the genetically engineered MSCs in a medium suitable for growth and propagation of the genetically engineered MSCs and harvesting a population of genetically engineered MSCs.

In one embodiment, the present disclosure provides a population of engineered mesenchymal stem cells described herein.

Examples of the MSC include, but are not limited to, an umbilical cord mesenchymal stem cells (UMSC), adipose derived mesenchymal stem cells (ADSC), or bone marrow mesenchymal stem cells (BMSC).

In one embodiment, the genetically engineered MSCs described herein comprise a U6 promoter operatively linked to the exogenous genes.

In some embodiments of the disclosure, the genetic alterations and/or exogenous genes are introduced to MSC by using a vector comprising genes for adipogenic differentiation, mitochondrial biogenesis and a brown adipocyte marker.

In some embodiments of the disclosure, the genetic alterations and/or exogenous genes are introduced to MSC by using a vector comprising PPARγ, PGC1a and UCP1 genes.

In some embodiments of the disclosure, the vector is a transposon vector.

In some embodiments of the disclosure, the genetic alterations and/or exogenous genes are introduced to MSCs by using clustered regularly interspaced short palindromic repeats (CRISPR)/nuclease.

In some embodiments of the disclosure, the vector comprises dCas9-VP64 and MS2-p65-HSF1 fragments.

In some embodiments of the disclosure, the vector comprises a U6 promoter operatively linked to the exogenous genes.

In some embodiments of the disclosure, the vector is an all-in-one CRISPR-based multigene activation plasmid constructed by steps of:
providing gRNA cloning vectors comprising recombination sites and PPARγ, PGC1α and UCP1, respectively, and
mixing the gRNA cloning vectors with a transposon backbone vector containing CRISPR/nuclease to generate all-in-one CRISPR-based multigene activation plasmid.

As embodied and broadly described herein, one another aspect of the disclosure is directed to a pharmaceutical composition comprising the genetically engineered MSC as described herein and optionally a pharmaceutically acceptable carrier.

As embodied and broadly described herein, one another aspect of the disclosure is directed to use of the pharmaceutical composition as described herein in the manufacture of a medicament for preventing, ameliorating and/or treating a metabolic disorder in a subject in need thereof. Alternatively, one another aspect of the disclosure is directed to a method for preventing, ameliorating and/or treating a metabolic disorder in a subject in need thereof comprising administering the pharmaceutical composition as described herein to the subject. Still alternatively, one another aspect of the disclosure is directed to the pharmaceutical composition as described herein for use in preventing, ameliorating and/or treating a metabolic disorder in a subject in need thereof.

Examples of the metabolic disorder include, but are not limited to diabetes, obesity, central obesity, insulin-resistance, hypertension, an insulin resistance disorder, hepatic steatosis, cardiac deficiency, ischemic cardiac disease, high blood pressure, inflammation, triglyceride dyslipidemia, HDL dyslipidemia, cholesterol dyslipidemia, elevated fasting plasma glucose, leptin dysregulation, adipon dysregulation, or cancer. In some embodiments of the disclosure, the metabolic disorder is metabolic dysfunction-associated steatotic liver disease (MASLD).

In some embodiments of the disclosure, the medicament is for improving insulin sensitivity, promoting efficient energy utilization, reshaping immune cell populations, and/or improving lipid accumulation, inflammation or fibrosis within liver.

In some embodiments of the disclosure, the effective amount ranges from about 1×10⁴cells to about 1×10⁸ cells; from about 2×10⁴ cells to about 8×10⁷ cells; from about 5×10⁴ cells to about 5×10⁷ cells; from about 8×10⁴ cells to about 2×10⁷ cells; from about 1×10⁵ cells to about 1×10⁷ cells; from about 2×10⁵ cells to about 8×10⁶ cells; from about 5×10⁵ cells to about 5×10⁶ cells; from about 8×10⁵ cells to about 2×10⁶ cells; about 1×10⁶ cells.

In some embodiments of the disclosure, the medicament is administrated by intravenous injection.

Many one of the attendant features and advantages of the present disclosure will become better understood with reference to the following detailed description considered in connection with the accompanying drawings.

### BRIEF DESCRIPTION OF DRAWINGS

FIGS. 1a and 1b show generation of one-step CRISPR-based multigene activation system. FIG. 1a shows that 3 gRNAs designed for different gene promoters can be cloned into 3 vectors. Using multiple-site Gateway cloning technology, all 3 gRNAs can be cloned into transposon vector together with CRISPR-based activation machinery (dCas9-VP64 and MS2-p65-HSF1) to create all-in-one plasmid. FIG. 1b shows that when all-in-one plasmid is delivered into cells and incorporated into DNA by transposase, CRISPR-SAM system will activate 3 different genes corresponding to 3 gRNAs at the same time.

FIGS. 2a and 2b show generation of BATMen using CRISPR-based multigene activation. FIG. 2a shows that Human mesenchymal stem cells were delivered by a vector expressed transposase and an all-in-one plasmid with or without 3 different gRNAs targeting UCP1, PPARγ and PGC1α (BATMen and CTL-hMSC). FIG. 2b shows the expression of MS2, UCP1, PPARγ and PGC1α in original hMSC, CTL-hMSC and BATMen.

FIGS. 3a to 3h show BATMen improved glucose tolerance and energy expenditure in MASLD mice. FIG. 3a shows scheme of cell therapy including CTL-hMSC and BATMen for MASLD treatment. FIG. 3b shows distribution of DiR-stained BATMen after tail vein injection. FIGS. 3c to 3e show that after 4 times of PBS or cell injection, body weight (FIG. 3c), lean mass (FIG. 3d), fat mass (FIG. 3e) of mice were measured. FIGS. 3f to 3h show that metabolic parameters including glucose tolerance test (GTT, FIG. 3f), insulin tolerance test (ITT, FIG. 3g), and energy expenditure (FIG. 3h) were determined in MASLD mouse models after 4 times of PBS or cell injection.

FIGS. 4a to 4d show that BATMen resolves the lipid accumulation, fibrosis and damage in liver of MASLD mice. FIG. 4a shows that Oil Red O and Masson's trichrome were used to stain lipid droplets and collagen fibers, respectively, in liver sections from normal diet mice and MASLD mice treated with hMSC or BATMen. (FIGS. 4b and 4c) The mRNA expression levels of genes, including Col3a1 (FIG. 4b) and TNFα (FIG. 4c), in the liver of mice. FIG. 4d show the ALT activity in the plasma of mice.

FIGS. 5a to 5d show that BATMen regulates immune cell infiltration in liver of MASLD mice. FIG. 5a shows using CyTOF to profile different immune cell populations in liver of normal diet mice and MASLD mice infected with hMSC or BATMen. FIG. 5b shows quantification of different immune cell population. FIG. 5c shows tSNE map for CD11b⁺ monocyte/macrophages in liver of mice with different treatments. FIG. 5d shows the expression of MASLD-associated microphages (NAM) markers including GPNMB, TREM2 and APOE in isolated non-parenchymal cells (NPC) from liver of mice.

### DETAILED DESCRIPTION OF THE INVENTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and claims of the present invention are approximate and can vary depending upon the desired properties sought by the present invention.

The term “a/an” should mean one or more than one of the objects described in the present invention. The term “and/or” means either one or both of the alternatives. The term “a cell” or “the cell” may include a plurality of cells.

The term “and/or” is used to refer to both things or either one of the two mentioned.

As used herein, the term “stem cell” refers to a cell in an undifferentiated or partially differentiated state that has the property of self-renewal and has the developmental potential to naturally differentiate into a more differentiated cell type, without a specific implied meaning regarding developmental potential (i.e., totipotent, pluripotent, multipotent, etc.). By self-renewal is meant that a stem cell is capable of proliferation and giving rise to more such stem cells, while maintaining its developmental potential. Accordingly, the term “stem cell” refers to any subset of cells that have the developmental potential, under particular circumstances, to differentiate to a more specialized or differentiated phenotype, and which retain the capacity, under certain circumstances, to proliferate without substantially differentiating.

The term “genetically engineered” or “genetic engineering” of cells means manipulating genes using genetic materials for the change of gene copies and/or gene expression level in the cell. The genetic materials can be in the form of DNA or RNA. The genetic materials can be transferred into cells by various means including viral transduction and non-viral transfection. After being genetically engineered, the expression level of certain genes in the cells can be altered permanently or temporarily.

As used herein, the term “exogenous” refers to any material introduced from or produced outside an organism, cell, tissue or system.

As used herein, the term “vector” is intended to refer to a nucleic acid molecule capable of transporting another nucleic acid to which it has been linked.

The term “expression vector” means the agent carrying foreign genes into cells for expression without degradation. The expression vector in the present invention can be plasmid, viral vectors, and artificial chromosomes.

The term “increased expression” herein means the increased expression of RNA or protein of the genes of interest in genetically engineered cells, when compared to the expression level of those genes in the non-engineered cell counterpart.

“Operatively linked” refer to two nucleic acid sequences that are related physically or functionally. For example, a promoter or regulatory DNA sequence is said to be “associated with” a DNA sequence that codes for an RNA or a protein if the two sequences are operatively linked, or situated such that the regulator DNA sequence will affect the expression level of the coding or structural DNA sequence.

As used herein, “recombining sites” (also referred to herein as “recombination sites”) refer to nucleic acid sequences comprising inverted palindromes separated by an asymmetric sequence at which a site-specific recombination can occur. Such recombining sites can include, but are not limited to, Lox (Sternberg et al. (1978) Cold Spring Harbor Symp. Quant. Biol. 43:1143-1146 and Hoess et al. (1990) In Nucleic Acids and Molecular Biology, Eds Eckstein and Lilley (Springer, Berlin), vol 4, pp 99-109) and FRT (reviewed in Kilby et al. (1993) Trends In Genetics, 9, 413-421).

The term “pharmaceutical composition” of this disclosure includes an effective amount of live cells to treat a metabolic disorder. The cell component may be a mixture of culture cells or an isolated population of cells, such as differentiated tissue cells, progenitor cells, and/or stem cells. The pharmaceutical composition of this disclosure is in liquid form or cell suspension buffer, and it may contain pharmaceutically acceptable excipients that stabilize the liquid suspension and help cell viability.

As used herein, the terms “treat,” “treating” and “treatment” are interchangeable, and encompass partially or completely preventing, ameliorating, mitigating, and/or managing a symptom, a secondary disorder, or a condition associated with the metabolic disorder. The term “treating” as used herein refers to the application or administration of the pharmaceutical composition of the present disclosure to a subject, who has a symptom, a secondary disorder, or a condition associated with the metabolic disorder, with the purpose of partially or completely alleviate, ameliorate, relieve, delay the onset of, inhibit the progression of, reduce the severity of, and/or reduce the incidence of one or more symptoms, secondary disorders or features associated with the metabolic disorder. Treatment may be administered to a subject who exhibits only early signs of such symptoms, disorder, and/or condition for the purpose of decreasing the risk of developing the symptoms, secondary disorders, and/or conditions associated with the metabolic disorder. Treatment is generally “effective” if one or more symptoms or clinical markers are reduced as that term is defined herein. Alternatively, a treatment is “effective” if the progression of a symptom, disorder, or condition is reduced or halted.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

The term “effective amount” as referred to herein designates the quantity of a component that is sufficient to yield a desired response. For therapeutic purposes, the effective amount is also one in which any toxic or detrimental effects of the component are outweighed by the therapeutically beneficial effects. An effective amount of an agent is not required to cure a disease or condition but will provide treatment for a disease or condition such that the onset of the disease or condition is delayed, hindered, or prevented, or the disease or condition symptoms are ameliorated. The effective amount may be divided into one, two, or more doses in a suitable form to be administered one, two, or more times throughout a designated time period. The specific effective or sufficient amount will vary with such factors as the particular condition being treated, the physical condition of the patient (e.g., the patient's body mass, age, or gender), the type of mammal or animal being treated, the duration of the treatment, the nature of concurrent therapy (if any), and the specific formulations employed and the structure of the compounds or its derivatives. Effective amounts may be expressed, for example, in grams, milligrams, or micrograms or as milligrams per kilogram of body weight (mg/Kg). Alternatively, the effective amount can be expressed in the concentration of the active component (e.g., the immunoconjugate of the present disclosure), such as molar concentration, mass concentration, volume concentration, molality, mole fraction, mass fraction, and mixing ratio. Persons having ordinary skills could calculate the human equivalent dose (HED) for the medicament (such as the present immunoconjugate) based on the doses determined from animal models. For example, one may follow the guidance for industry published by the US Food and Drug Administration (FDA) entitled “Estimating the Maximum Safe Starting Dose in Initial Clinical Trials for Therapeutics in Adult Healthy Volunteers” in estimating a maximum safe dosage for use in human subjects.

As interchangeably used herein, the terms “individual,” “subject,” and “patient,” refer to an animal, preferably a mammal. Examples of the subject include human, non-human primates, rodents, guinea pigs, rabbits, sheep, pigs, goats, cows, horses, dogs and cats.

As used herein, the term “in need of treatment” refers to a judgment made by a caregiver (e.g. physician, nurse, nurse practitioner, or individual in the case of humans; veterinarian in the case of animals, including non-human mammals) that a subject requires or will benefit from treatment. This judgment is made based on a variety of factors that are in the realm of a care giver's expertise, but that includes the knowledge that the subject is ill, or will be ill, as the result of a condition that is treatable by the compounds of the present disclosure.

Approximately 23% of the adult population is currently affected by metabolic syndrome, and its prevalence is increasing worldwide. Consequently, there is a high demand for therapeutic targets that can reduce obesity and insulin resistance, the two hallmark features of metabolic syndrome. Adaptive thermogenesis is a physiological process that generates heat by releasing stored chemical energy in response to environmental cues. This process is critical because even minor differences in energy consumption through adaptive thermogenesis can significantly impact systemic metabolism over time. As a result, targeting adaptive thermogenesis is a promising strategy for preventing and treating metabolic diseases. Thermogenesis primarily occurs in thermogenic fat, namely brown and beige/brite adipose tissue (BAT), making it an attractive target for intervention.

As embodied and broadly described herein, one aspect of the disclosure is directed to a population of genetically engineered mesenchymal stem cells (MSC). In some embodiments of the disclosure, the MSC comprises genetic alterations and/or exogenous genes that increase gene expressions for adipogenic differentiation, mitochondrial biogenesis and a brown adipocyte marker. In some embodiments of the disclosure, the MSCs comprises exogenous genes of comprising adipogenic differentiation, mitochondrial biogenesis and a brown adipocyte marker, wherein the gene expressions of adipogenic differentiation, mitochondrial biogenesis and a brown adipocyte marker are increased.

For example, the MSCs described herein may constitute at least 70 percent of the total cells in the cell population, present with greater percentages, e.g., at least 85, 90, 95 or 99 percent, being preferred.

In some embodiments of the disclosure, the genetically engineered engineer mesenchymal stem cells endow themselves with brown-like functions and are referred as BAT-like mesenchymal stem cells (BATMen). Brown adipose tissue (BAT) and its related beige fat are specialized in energy expenditure and fuel utilization. Moreover, BAT exhibits endocrine function by communicating with multiple cells/organs to modulate whole-body metabolism through secreted factors. A growing body of evidence indicates that BAT activation enhances glucose homeostasis, insulin sensitivity, and inflammation resolution to counteract the onset of type 2 diabetes and cardiovascular diseases in clinical settings. Significantly, the administration of BATMen substantially enhances glucose homeostasis by improving insulin sensitivity and promoting efficient energy utilization. Additionally, BATMen effectively alleviates MASLD-related inflammation by reshaping immune cell populations within the liver microenvironment. Collectively, these findings underscore the therapeutic promise of BATMen cell therapy in addressing MASLD development.

In some embodiments of the disclosure, the genetic alterations increase gene expression of peroxisome proliferator-activated receptor gamma (PPARγ). PPARγ is a master regulator of adipogenesis, the process by which preadipocytes differentiate into mature adipocytes. Its activation induces the expression of genes involved in fat storage, lipid metabolism, and insulin sensitivity. PPARγ promotes the conversion of mesenchymal stem cells into adipocytes by regulating the expression of genes like C/EBPα and FABP4, which are crucial for lipid uptake and fat storage.

In some embodiments of the disclosure, the genetic alterations increase gene expression of peroxisome proliferator-activated receptor-γ coactivator 1-α (PGC1α). PGC-1α is a transcriptional coactivator that induces mitochondrial biogenesis and thermogenesis. Through coactivating transcription factors like PPARγ and nuclear respiratory factors (NRFs), PGC-1α increases mitochondrial density and the expression of uncoupling protein 1 (UCP1), which is the hallmark of thermogenic cells. Thus, it plays a pivotal role in the browning of white adipose tissue, converting white fat cells into beige or brown-like adipocytes. Browning is characterized by the development of thermogenic, mitochondria-rich fat cells that can burn energy through non-shivering thermogenesis.

In some embodiments of the disclosure, the genetic alterations increase gene expression of mitochondrial uncoupling protein 1 (UCP1). Mitochondria in thermogenic fat cells express high levels of uncoupling protein 1 (UCP1), which uncouples oxidative phosphorylation, leading to the dissipation of a significant portion of the energy generated by the proton motive force across the inner mitochondrial membrane as heat. However, the benefits of activating thermogenic fat cells are not limited to energy dissipation. Brown and beige fat cells can also produce and release factors such as peptides, lipids, and other metabolites that act as endocrine, paracrine, or autocrine agents to regulate metabolism.

The mesenchymal stem cell according to the disclosure can be obtained from different sources, preferably from umbilical cord, adipose tissue or bone marrow. According to different sources, the mesenchymal stem cell is an umbilical cord mesenchymal stem cell (UMSC), adipose derived mesenchymal stem cell (ADSC), and bone marrow mesenchymal stem cell (BMSC). In some embodiments of this disclosure, the MSC are isolated and purified from the umbilical cord, and referred to as “umbilical MSC” or “UMSC.” In some embodiments, it is established that the UMSC in this disclosure expresses the same selection of surface markers as the MSC isolated from other bodies, and demonstrates comparable activities.

In some embodiments of the disclosure, the MSC comprises genetic alterations that modify nucleic acid sequences within cells to generate genetically engineered MSCs. Exemplary technologies of genetic alteration include, but are not limited to homologous recombination, knock-in, ZFNs (zinc finger nucleases), TALENs (transcription activator-like effector nucleases), CRISPR (clustered regularly interspaced short palindromic repeats)/Cas9, and other site-specific nuclease technologies. These techniques enable double-strand DNA breaks at desired locus sites. These controlled double-strand breaks promote homologous recombination at the specific locus sites. This process focuses on targeting specific sequences of nucleic acid molecules, such as chromosomes, with endonucleases that recognize and bind to the sequences and induce a double-stranded break in the nucleic acid molecule. The double-strand break is repaired either by an error-prone non-homologous end-joining (NHEJ) or by homologous recombination (HR).

The term “CRISPR/Cas” or “clustered regularly interspaced short palindromic repeats system” or “CRISPR” interchangeably refers to DNA loci containing short repetitions of base sequences. Each repetition is followed by short segments of spacer DNA from previous exposures to a virus or plasmid. Bacteria and archaea have evolved adaptive immune defenses termed CRISPR/CRISPR-associated (Cas) systems that use short RNA to direct degradation of foreign nucleic acids. In bacteria, the CRISPR system provides acquired immunity against invading foreign DNA via. RNA-guided DNA cleavage. To direct Cas9 to cleave sequences of interest, crRNA-tracrRNA fusion transcripts, hereafter referred to as “guide RNAs” or “gRNAs” may be designed, from human U6 polymerase III promoter. CRISPR/CAS mediated genome editing and regulation, highlighted its transformative potential for basic science, cellular engineering and therapeutics. In the type II CRISPR/Cas system, short segments of foreign DNA, termed “spacers” are integrated within the CRISPR genomic loci and transcribed and processed into short CRISPR RNA (crRNA). These crRNAs anneal to trans-activating crRNAs (tracrRNAs) and direct sequence-specific cleavage and silencing of invading DNA by Cas proteins. Recent work has shown that target recognition by the Cas9 protein requires a “seed” sequence within the crRNA and a conserved dinucleotide-containing protospacer adjacent motif (PAM) sequence upstream of the crRNA-binding region.

In some embodiments of the disclosure, the genetic alterations and/or exogenous genes are introduced to MSCs by using a vector comprising genes for adipogenic differentiation, mitochondrial biogenesis and a brown adipocyte marker. In some embodiments of the disclosure, the genetic alterations are introduced to MSCs by using a vector comprising PPARγ, PGC1α and UCP1 genes. In some embodiments of the disclosure, the genetic alterations refers to transferring an exogenous gene or gene fragment into the mesenchymal stem cells so that they can express the exogenous gene or gene fragment. In another aspect, this modification is a stable modification, and the expression may be persistent or inducible.

In some embodiments of the disclosure, the vector is a transposon vector.

In some embodiments of the disclosure, the vector comprises dCas9-VP64 and MS2-p65-HSF1 fragments.

In some embodiments of the disclosure, the vector comprises a U6 promoter operatively linked to the exogenous genes.

In some embodiments of the disclosure, the vector is an all-in-one CRISPR-based multigene activation plasmid constructed by steps of:
providing gRNA cloning vectors comprising recombination sites and PPARγ, PGC1α and UCP1, respectively, and

mixing the gRNA cloning vectors with a transposon backbone vector containing CRISPR/nuclease to generate all-in-one CRISPR-based multigene activation plasmid.

In one embodiment, the genetically modified MSCs described herein comprise the promoter resulting in constitutive expression of the exogenous nucleic acid.

In one embodiment, the genetically modified MSCs described herein comprise a U6 promoter operatively linked to the exogenous genes.

The population of genetically engineered MSCs described herein can be prepared by preparing a vector comprising all-in-one CRISPR-based multigene activation plasmid, wherein the plasmid is prepared by providing gRNA cloning vectors comprising recombination sites and PPARγ, PGC1α and UCP1, respectively, and mixing the gRNA cloning vectors with a transposon backbone vector containing CRISPR/nuclease to generate all-in-one CRISPR-based multigene activation plasmid; transfecting MSCs with the vectors to generate genetically engineered MSCs, culturing the genetically engineered MSCs in a medium suitable for growth and propagation of the genetically engineered MSCs and harvesting a population of genetically engineered MSCs.

Any medium and culture condition suitable for growth and propagation of the genetically engineered MSCs can be used in the present disclosure.

As embodied and broadly described herein, one another aspect of the disclosure is directed to a pharmaceutical composition comprising the genetically engineered MSC as described herein and optionally a pharmaceutically acceptable carrier. According to the disclosure, in addition to the population of genetically engineered MSCs, the above composition may contain one or more inactivated carriers that are permitted pharmaceutically. Examples of the inactivated carriers include preservative, solubilizer, stabilizer, etc. The composition may be used for non-oral administration, for example intravenous, subcutaneous, intra-peritoneal administration or topical application. A dosage of the cell population may vary in accordance with kind of disease, degree of seriousness of disease, administration route, or weight, age and sex of subject.

In some embodiments, the mesenchymal stem cells according to the disclosure are contained in an injectable preparation. The injectable preparation may be prepared by publicly known methods. For example, the injectable preparations may be prepared, e.g., by dissolving, suspending or emulsifying the pharmaceutical composition in a sterile aqueous medium or an oily medium conventionally used for injections. Examples of the aqueous medium for injections include physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. Examples of the oily medium include sesame oil, soybean oil, etc., which may be used in combination with a solubilizing agent such as benzyl benzoate, benzyl alcohol, etc. The injection thus prepared is preferably filled in an appropriate ampoule.

Route of administration of MSC in this invention depends on the tissue or organs in need of treatment. In some embodiments with the subjects having myocardial infarction, the route of administering MSCs can be intravenous, intraarterial, or the combination thereof. Solutions containing the cells can be prepared in suitable diluents such as water, ethanol, glycerol, liquid polyethylene glycol(s), various oils, and/or mixtures thereof, and others known to those skilled in the art.

As embodied and broadly described herein, one another aspect of the disclosure is directed to use of the pharmaceutical composition as described herein in the manufacture of a medicament for preventing, ameliorating and/or treating a metabolic disorder in a subject in need thereof. Alternatively, one another aspect of the disclosure is directed to a method for preventing, ameliorating and/or treating a metabolic disorder in a subject in need thereof comprising administering the pharmaceutical composition as described herein to the subject. Still alternatively, one another aspect of the disclosure is directed to the pharmaceutical composition as described herein for use in preventing, ameliorating and/or treating a metabolic disorder in a subject in need thereof.

Examples of the metabolic disorder include, but are not limited to diabetes, obesity, central obesity, insulin-resistance, hypertension, an insulin resistance disorder, hepatic steatosis, cardiac deficiency, ischemic cardiac disease, high blood pressure, inflammation, triglyceride dyslipidemia, HDL dyslipidemia, cholesterol dyslipidemia, elevated fasting plasma glucose, leptin dysregulation, adipon dysregulation, or cancer. In some embodiments of the disclosure, the metabolic disorder is metabolic dysfunction-associated steatotic liver disease (MASLD).

In some embodiments of the disclosure, the medicament is for improving insulin sensitivity, promoting efficient energy utilization, reshaping immune cell populations, and/or improving lipid accumulation, inflammation or fibrosis within liver.

The following Examples are provided to elucidate certain aspects of the present invention and to aid those skilled in the art in practicing this invention. These Examples are in no way to be considered to limit the scope of the invention in any manner. Without further elaboration, it is believed that one skilled in the art can, based on the description herein, utilize the present invention to its fullest extent. All publications cited herein are hereby incorporated by reference in their entirety.

### EXAMPLE

### Materials and Methods

### Preparation, Isolation, and Characterization of Human UMSCs

The collected human umbilical cord tissues were washed three times with Ca²⁺- and Mg²⁺-free PBS (DPBS, Life Technology). They were mechanically cut by scissors in a midline direction, and the vessels of the umbilical artery, vein, and outlining membrane were dissociated from Wharton's jelly (WJ). The jelly content was then extensively cut into pieces smaller than 0.5 cm³, treated with collagenase type 1 (Sigma, St Louis, USA), and incubated for 3 h in 5% CO₂ at 37° C. The explants then were cultured in NutriStem® MSC XF Medium with NutriStem® XF Supplement Mix (Sartorius, Germany) and 5% UltraGROTM-Advanced-PURE Cell Culture Supplement (AventaCell Biomedical, USA) and antibiotics in 5% CO₂ at 37° C. They were left undisturbed for 5-7 days to allow migration of the cells from the explants. The cellular morphology of umbilical cord-derived mesenchymal stem cells (UMSCs) became homogenously spindle-shaped in cultures after 4-8 passages. The specific surface molecules of cells from WJ were characterized by flow cytometry. The cells were detached with TrypLE™ Select Enzyme (Gibco, USA), washed with PBS, and incubated with the respective antibodies including CD13, CD29, CD44, CD73, CD90, CD105, CD166, CD49b, CD1d, CD3, CD10, CD14, CD31, CD34, CD45, CD49d, CD56, CD117, HLA-ABC, and HLA-DR, conjugated with fluorescein isothiocyanate (FITC) or phycoerythrin (PE) (BD Biosciences, USA). Subsequently, cells were analyzed using a Becton Dickinson flow cytometer and the FlowJo v.7.6 software to characterize the markers of UMBCs.

### Generation of Genetically Modified UMSCs

To deliver CRISPR-MAP system to UMSCs, the cells were electroporated using a Nucleofector™ 2b Device (Cat. No. AAB-1001; Lonza Inc.) using the program Y-001 (HMEC, High Efficiency). Briefly, 5×10⁵ UMSC/cuvette was resuspended in Human Mesenchymal Stem Cell Nucleofector™ Kit (Cat. No. VPE-1001; Lonza Inc.) along with 2.5 μg transposon-based all-in-one CRISPR-MAP plasmid and 2.5 μg Super PiggyBac transposase expression plasmid. After electroporation, the cells were incubated in NutriStem® MSC XF Medium with NutriStem® XF Supplement Mix and 5% UltraGRO™-Advanced-PURE Cell Culture Supplement and cultured in T25 flasks. After 3 days of incubation, Blasticidine (5 μg/mL) was added to the culture media to select the cells with transposons encoding the target genes.

### Adipogenic Differentiation of UMSCs

For adipocyte differentiation, cells were grown in DMEM with 10% FBS for 3 days until reaching confluence and then treated with the adipogenic induction medium containing 10% FBS, 0.5 mM isobutylmethylxanthine, 0.1 μM dexamethasone, 0.5 μM human insulin, 2 nM T3, 30 μM indomethacin, 17 μM pantothenate, 33 μM biotin (Sigma-Aldrich, Dallas, TX) for another 12 days.

### Gene Expression

Total RNA was extracted from cells or tissues with Trizol and purified using a spin column kit (Zymo Research). RNA (1 μg) was reverse transcribed with a high-capacity complementary DNA (cDNA) reverse transcription kit (Applied Biosystems). Real-time PCR was performed in a solution containing cDNA (8 ng), forward and reverse oligonucleotide primers (300 nM each) and SYBR green PCR Master Mix (Roche). Fluorescence was determined and analyzed in an ABI 7900 sequence detection system (Applied Biosystems). 18S rRNA expression were used to normalize gene expression in human and mouse cells, respectively.

### Protein Expression

Cells were scraped from tissue-culture plates with RIPA buffer. After centrifugation (12,000 g, 15 min), protein lysates were harvested and stored at −80° C. until further use. Protein concentrations were determined by using the Pierce BCA kit (Life Technologies), according to the manufacturer's instructions. For immunoblots, lysates were diluted into Laemmli buffer, boiled, and loaded onto 4-15% Tris gels for SDS-PAGE. After complete separation of the proteins, they were transferred onto a PVDF membrane (Amersham Biosciences) and were blocked in western blocking buffer (Roche). Primary antibodies were applied in blocking buffer over night at 4° C. After washing 3 times for 10 min with TBS-T, secondary antibodies were applied for 1 h in blocking buffer. Membranes were washed again 3 times for 10 min in TBS-T and developed using chemiluminescence (ThermoFisher). For quantification of scanned immunoblots, integrated pixel density of interested bands was measured using ImageJ software. Total actin was used as an endogenous control for normalization.

### Animals

All animal experiments adhered to the relevant ethical regulations governing the use of small rodents. C57BL6J mice (Stock no. 000664) from the Jackson Laboratories. Mice had ad libitum access to food and water and were maintained on a normal chow diet (Mouse Diet 9F 5020, PharmaServ) containing 22% of calories from fat, 23% from protein, and 55% from carbohydrates. For MASLD mouse model, mice were fed a high fat diet containing 60 kcal % from fat (cat. no. D12492, Research Diets) and 30% fructose water. Prior to being euthanized, the mice were transferred to clean cages that were devoid of any food or feces in the hoppers or at the bottom and underwent a fasting period of 6 hours. Mice were anesthetized using inhalation of isoflurane (cat. no. NDC 66794-017-25, Piramal Critical Care), then blood was collected from the tails of mice to determine fasting glucose concentrations using an Infinity Blood Glucose Meter (US Diagnostics). Additional blood samples were obtained by cardiopuncture, subsequently plasma was separated by centrifugation at 4° C. and stored at −80° C. until future analysis of ALT activity. The BAT, pgWAT, scWAT, liver, and quadriceps muscle were harvested, weighed, then snap-frozen in liquid nitrogen and stored at −80° C. until further analysis.

### Intravenous Transplantation of CRISPR-Engineered UMSCs

After 6 weeks high fat diet and high fructose water feeding, male mice were anesthetized with chloral hydrate (0.4 g/kg, IP injection) and treated with 5×10⁵ cells through an intravenous route. Regarding the experimental animal randomization, we applied the computer-generated numbers/sampling methods to achieve randomization for each treatment group. The control animals were administered PBS only. Because of the immunosuppressive characteristics of mesenchymal stem cells, rat hosts did not receive any immunosuppressive medication.

### Biodistribution of UMSC in the Mouse Model

To evaluate the homing effect of CRISPR-engineered UMSCs, the biodistribution of the cells following intravenous implantation was assessed using the IVIS imaging system. The cells were labeled with 1 mg/mL of 1,1′-Dioctadecyl-3,3,3′,3′-Tetramethylindotricarbocyanine Iodide (DiR), a near-infrared fluorescent, lipophilic carbocyanine dye (Thermo Fisher Scientific, USA), for 60 minutes at room temperature. After labeling, the cells were washed with PBS and centrifuged at 3,000×g for 10 minutes at 4° C. The DiR-labeled UMSCs were then resuspended in sterile 1× PBS and administered intravenously into mice. One day post-injection, the mice were sacrificed, and various tissues were immediately collected for imaging using the IVIS system.

### Measurement of Body Lean and Body Fat

Body composition, including lean mass and fat mass, was measured using a dual-energy X-ray absorptiometry (DEXA) scanner. Prior to the scan, the machine was calibrated according to the manufacturer's instructions. Whole-body scans were conducted using the DEXA system's body composition mode, which uses two low-energy X-ray beams to differentiate between bone, lean tissue, and fat tissue based on their attenuation properties. The scan duration ranged from 5 to 15 minutes. The resulting data were analyzed using the scanner's software, which provided detailed measurements of total body fat percentage, lean body mass, and regional fat distribution.

### Indirect Calorimetry

Mice were individually housed in metabolic cages of a Comprehensive Lab Animal Monitoring System (CLAMS) at room temperature. After a 12 h acclimation period, animals were monitored for 24 h in order to obtain measurements for the volume of oxygen consumption (VO2), the volume of carbon dioxide production (VCO2) and respiratory exchange ratio (RER), which was calculated as the ratio of total VCO2 produced to total VO2 consumed, heat production, activity and accumulated food intake.

### Glucose and Insulin Tolerance Tests

For the glucose tolerance test (GTT), animals were fasted for 6 hours (7 AM to 1 PM) with free access to drinking water. A baseline blood sample was collected from the tail of fully conscious mice, followed by an i.p. injection of glucose (2.0 g/kg body weight), and blood was taken from the tail at 15, 30, 60, and 120 minutes after injection. For the insulin tolerance test (ITT), animals were fasted for 6 hours (7 AM to 1 PM) with free access to drinking water. Baseline blood samples were collected from the tail of fully conscious mice. Insulin (1 U/kg body weight) (HumulinO; Eli Lilly) was administered by i.p. injection, and blood samples were taken from the tail at 15, 30, 60, and 90 minutes after injection. Glucose concentrations were determined from blood using an InfinityO Blood Glucose Meter (US Diagnostics).

### Histological Analysis

Tissues were fixed for 24 hours in 10% formalin and embedded in paraffin. Paraffin blocks were cut into 5 μm sections and stained with hematoxylin and eosin (H&E). The sections were deparaffinized and rehydrated. Antigen retrieval was performed using a modified citrate buffer (Dako Target Retrieval Solution, Agilent). Blocking was carried out using blocking serum, followed by incubation with the primary antibodies overnight at 4° C. On the following day, the slides were washed with PBS and incubated with secondary antibodies at a 1:200 dilution for one hour. After a hematoxylin counterstain (S-3309, Agilent), the slides were mounted and imaged.

### Oil Red O Staining

The slides were washed twice with PBS and subsequently fixed with 10% buffered formalin for 15 min at room temperature. After fixation, the cells were stained with a working solution of filtered Oil Red O. This working solution was prepared by combining 3 parts of 0.5% Oil Red O in isopropanol with 2 parts of water. The staining process was conducted for 1 hour at room temperature. Following the staining, the cells were washed several times with distilled water and then subjected to visualization.

NPC Isolation from Liver

Liver non-parenchymal cells (NPCs) were isolated using a two-step pronase/collagenase digestion protocol. Briefly, the liver was perfused in situ with calcium-free Hank's Balanced Salt Solution (HBSS) containing 0.2 mg/mL EDTA. This was followed by sequential perfusion with 0.4 mg/mL pronase (Sigma, P5147) and 0.2% collagenase type II (Worthington, LS004196). The liver was then minced and further digested in HBSS containing 0.2% collagenase type II, 0.4 mg/mL pronase, and 0.1 mg/mL DNase I (Roche, R104159001) in a shaking water bath at 37° C. for 20 minutes. To terminate digestion, DMEM supplemented with 10% serum was added. The resulting liver cell suspension was centrifuged at 50×g for 3 minutes to remove hepatocytes and then passed through a 30 μm nylon cell strainer. Remaining red blood cells were lysed using 0.8% NH₄Cl.

### Phenotyping of Hepatic Lymphocytes Using Cytometry by Time-of-Flight (CyTOF)

Hepatic lymphocytes were phenotyped following the isolation of NPCs from mouse livers. Custom metal-conjugated antibodies were used to stain cell surface markers for profiling lymphocyte subtypes. Staining was performed using the Maxpar Cytoplasmic/Secreted Antigen Staining with Fresh Fix kit (Fluidigm, USA) according to the manufacturer's instructions. Briefly, cells were washed with PBS and stained with cisplatin to assess viability. The NPCs were then labeled with six surface markers included in the Maxpar Mouse Spleen/Lymph Node Basic Phenotyping Panel Kit (Fluidigm, USA). After surface marker staining, the cells were fixed with 1.6% formaldehyde and stained with Cell-ID Intercalator-Ir. Before data acquisition, the cells were washed and resuspended at a concentration of 1×10⁶ cells/mL in Cell Acquisition Solution (Fluidigm, USA). Calibration beads were added at a 1:10 ratio by volume for normalization. The stained cells were filtered into strainer-capped tubes and analyzed using the Helios mass cytometry platform.

### Statistics

All statistics were calculated using Microsoft Excel and GraphPad Prism. Unpaired Student's t tests were performed to compare only 2 groups. One-way and two-way ANOVA followed by Tukey's post-hoc tests were performed for multiple comparisons. Correlations were established based on Spearman's correlation tests, and the Spearman's correlation coefficient was provided. Significance of ANCOVA analysis was calculated directly by the Energy Expenditure Analysis Webpage of MMPC. All experiments involving mice were conducted with comparisons and statistical analysis within littermates. P values less than 0.05 were considered statistically significant.

### Example 1

### Establish CRISPR-based Multigene Activation Platform (CRISPR-MAP)

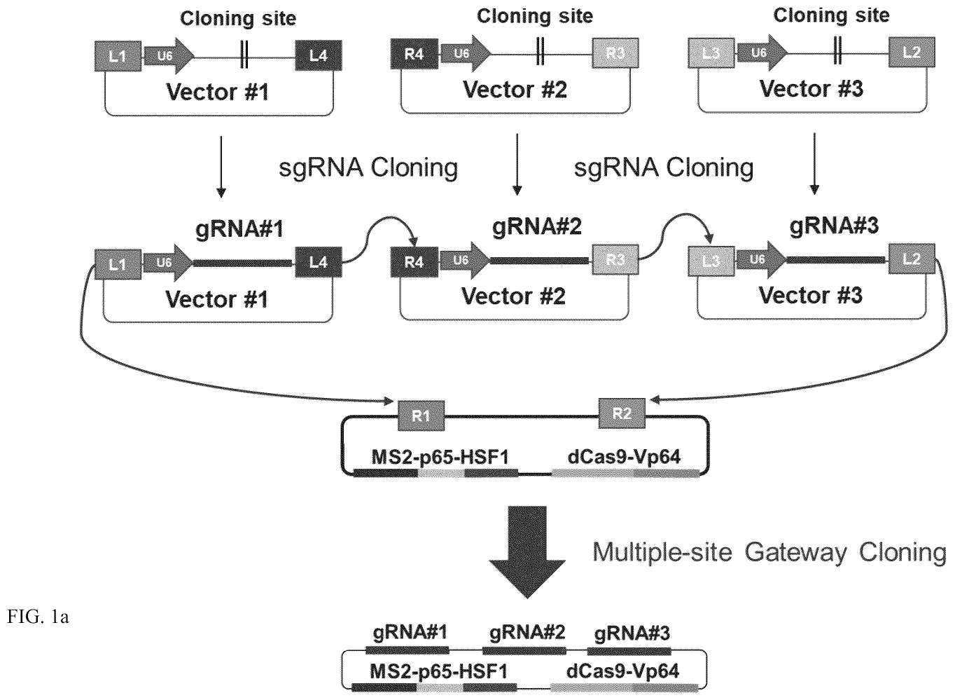

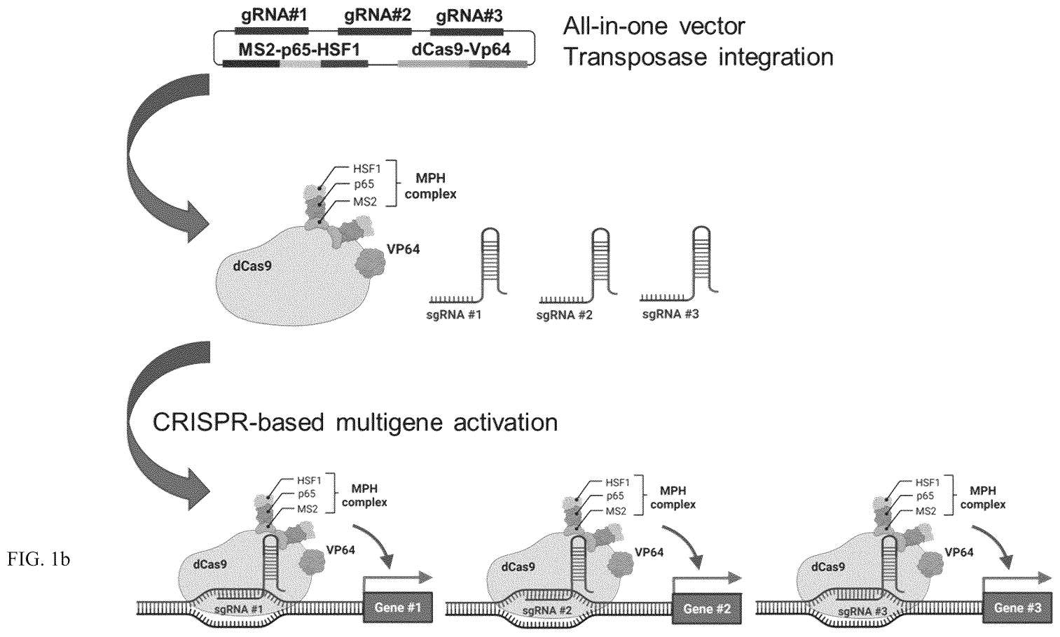

To achieve multigene activation in MSCs to engineer it to multifunctional MSCs, we established the CRISPR-based Multigene Activation Platform (CRISPR-MAP), which can activate multigene at the same time once delivering multiple gRNAs. First, we created 3 individual gRNA cloning vectors which were incorporated with the specific LR sequences for following Multiple-site Gateway Cloning. After cloning the specific gRNA into 3 vectors respectively, all 3 vectors were mixed with Transposon backbone vector containing with CRISPR-based activation system (dCas9-VP64 and MS2-p65-HSF1). By adding Multiple-Gateway Cloning enzyme, 3 gRNAs would be cloned into Transposon backbone to generate all-in-one CRISPR-based multigene activation plasmid (FIG. 1a). After delivering the all-in-one plasmid combined with transposase into cells, CRISPR-SAM system with 3 different gRNA would be integrated into DNA and constitutively activate 3 different genes (FIG. 1b).

### Example 2

### Generate BAT-Like Mesenchymal Stem Cells Using CRISPR-MAP Method

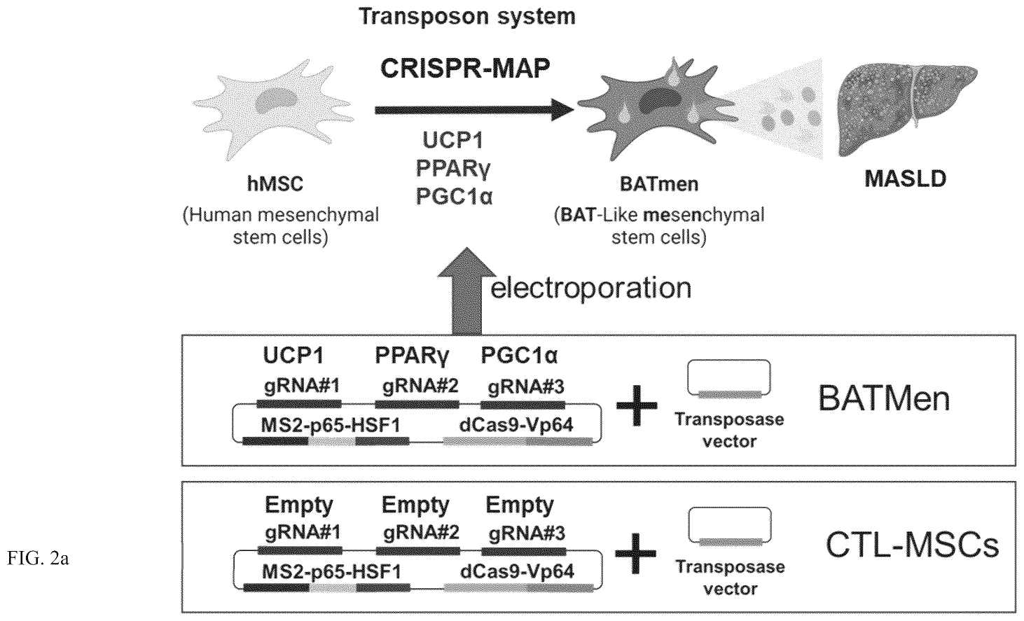

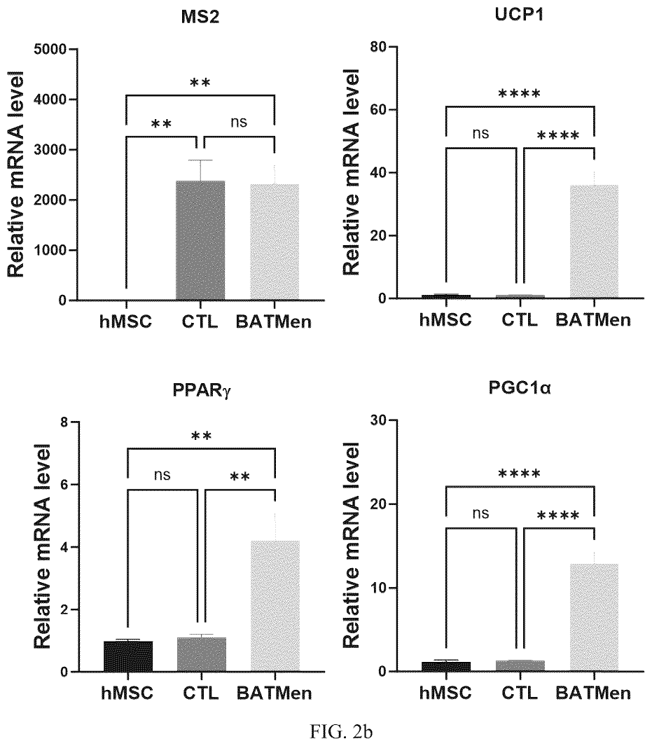

Given that the function of BAT serves as potential therapeutic roles for the MASLD treatment, we aimed to engineer MSCs to acquire the BAT function via CRISPR-MAP and named the engineered MSCs as BAT-like Mesenchymal stem cells (BATMen). To establish BATMen, we targeted 3 different genes involved in adipogenic differentiation (PPARy), mitochondrial biogenesis (PGC1α) and brown marker (UCP1). After co-transfection with a vector expressing transposase and all-in-one plasmid with or without 3 gRNAs, we obtained BATMen and CTL-hMSC (FIG. 2a). The results showed both cells expressed CRISPR-SAM system (MS2) compared to the parental hMSC. Importantly, BATMen expressed high levels of PPARγ, PGC1α and UCP1 based on the delivery of 3 gRNAs (FIG. 2b).

### Example 3

### BATMen Therapy Alleviates Metabolic Dysregulation of MASLD Mouse Model

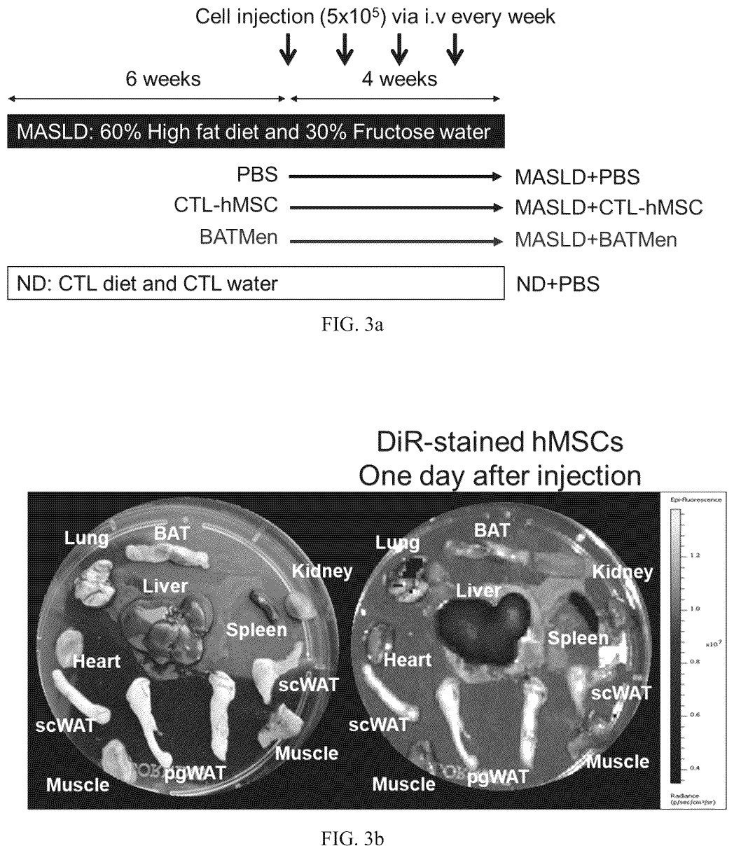

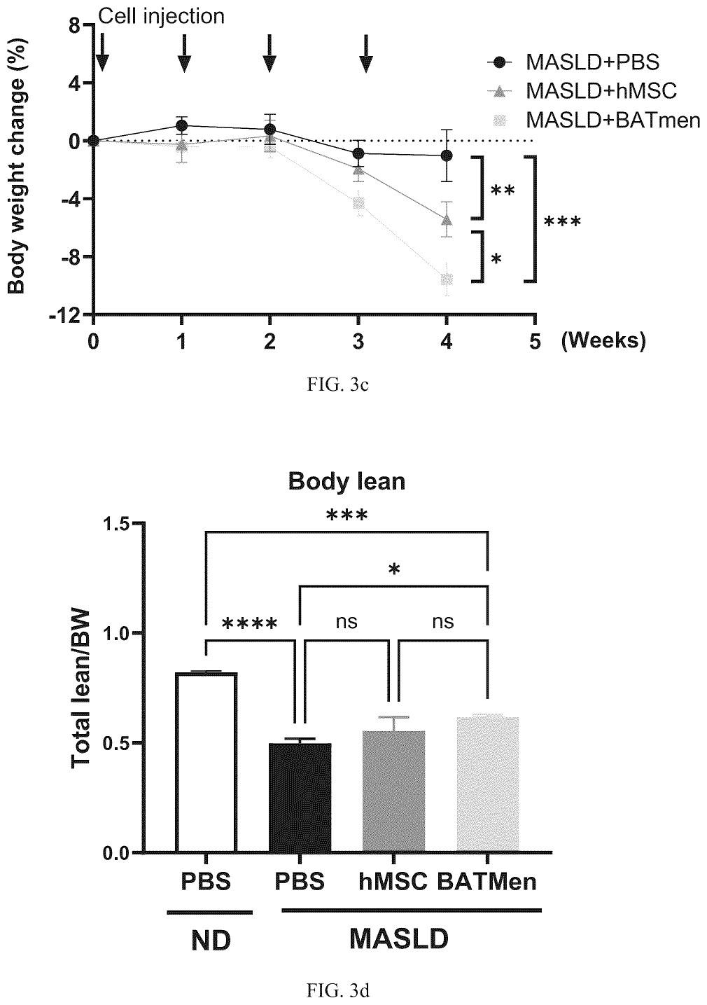

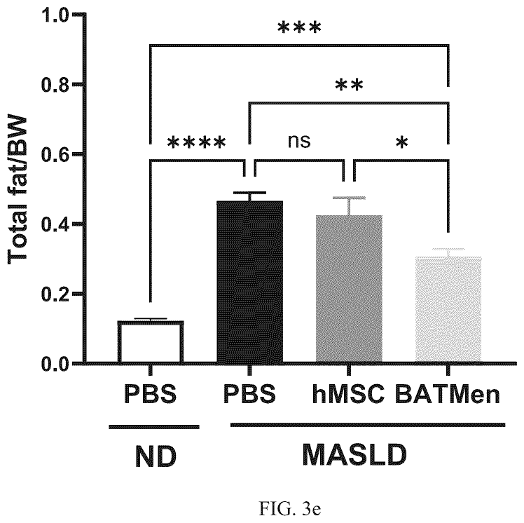

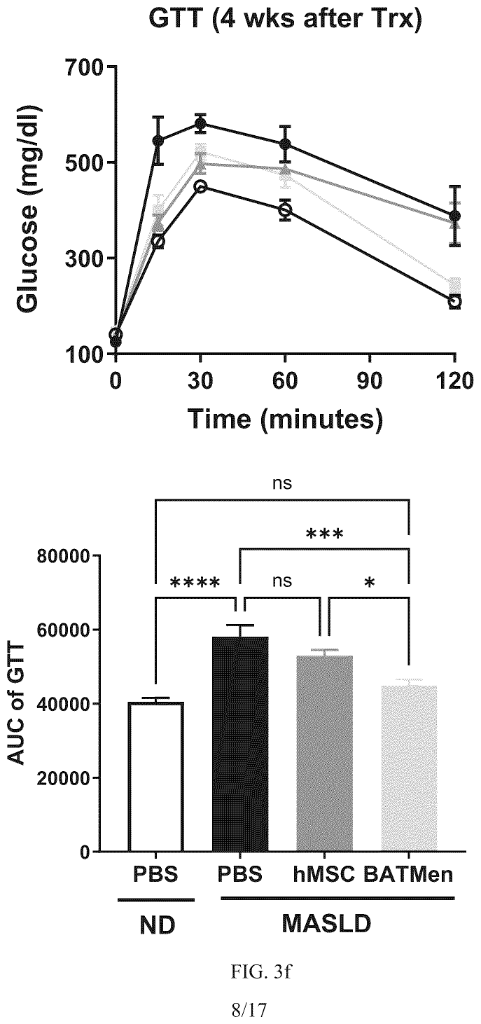

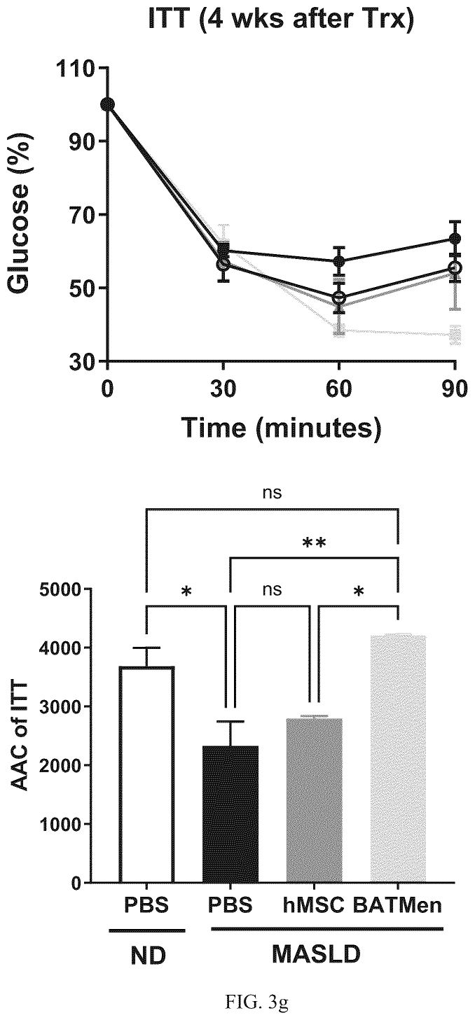

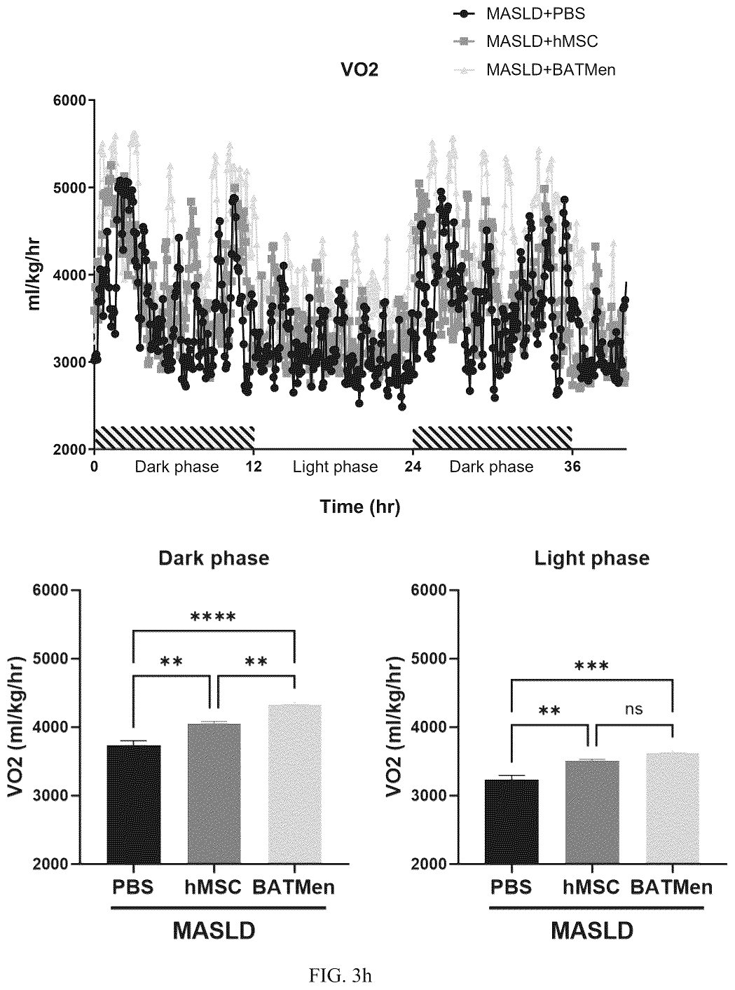

To model MASLD disease, C57BL/6 mice will be fed a high-fat diet (HFD; 20% protein, 60% fat, and 20% carbohydrates, Research diets D12492) and a 30% (wt/v) fructose solution in drinking water (HFW) for 10-14 weeks, following a previously established protocol (Softic, S., et al., Divergent effects of glucose and fructose on hepatic lipogenesis and insulin signaling. J Clin Invest, 2017. 127 (11): p. 4059-4074). For the allogeneic cell therapy protocol, PBS or CTL-hMSC or BATMen will be injected via the tail vein of the mice after 6 weeks of HFD and HFW. Cell injection (5×10⁵ cells) will be performed once a week for a total of 10 weeks (FIG. 3a). After receiving 4 rounds of cell therapy, the mice will undergo metabolic characterization using the methods described below. To know the biodistribution of BATMen after tail vein injection, we pre-stain BATMen with DiR. After one day of injection, the red signal can be detected mainly in liver, and some of signals in spleen and lung (FIG. 3b). It indicated BATMen can target the liver and is able to regulate MASLD microenvironment.

Intriguingly, 4 rounds of BATMen cell therapy could decrease body weight gain upon the HFD treatment (FIG. 3c). The results also showed increase of body lean and decrease of body fat in MASLD mice treated with BATMen (FIGS. 3d and 3e). In addition, BATMen improved the glucose tolerance and insulin sensitivity monitoring by GTT and ITT (FIGS. 3f and 3g). Moreover, BATMen activated energy expenditure to consume more energy (FIG. 3h). Taken together, BATMen cell therapy can counteract dysregulation of glucose and energy metabolism in MASLD mice.

### Example 4

### BATMen Resolve the Lipid Accumulation, Inflammation, Fibrosis Within Liver of MASLD Mice

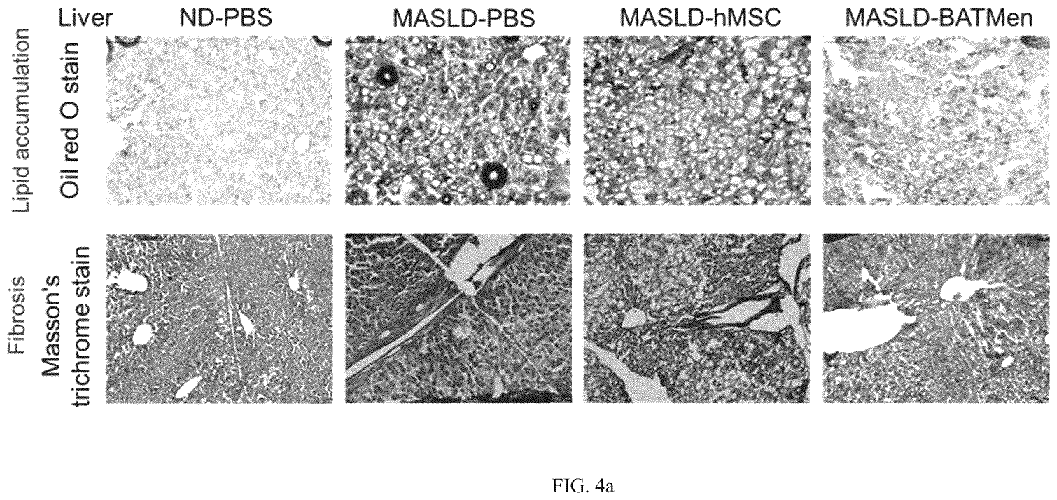

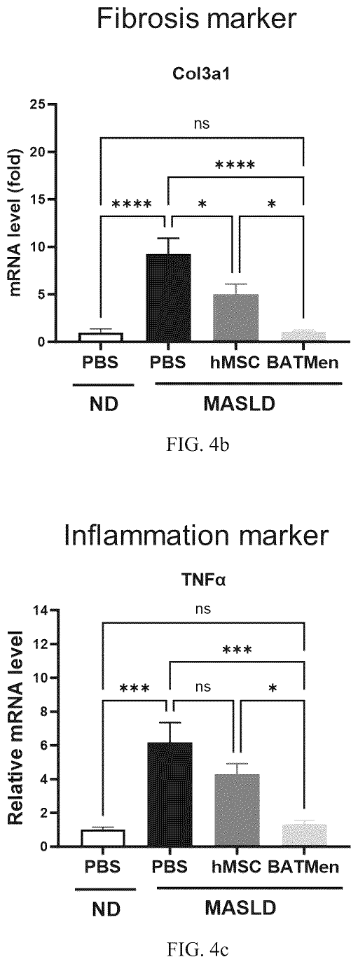

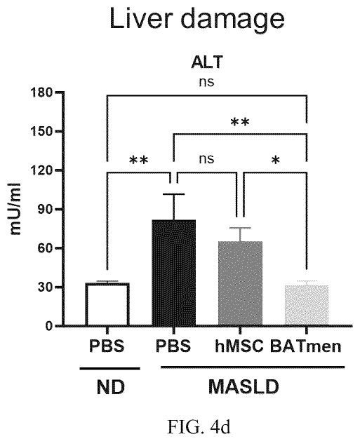

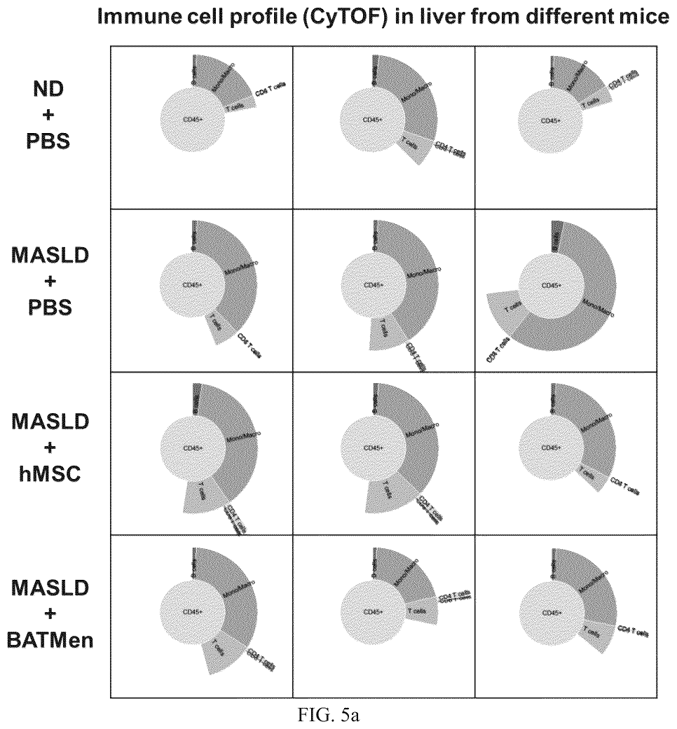

To investigate whether BATMen ameliorates the pathogenesis of MASLD, we examined the histological, biochemical, and molecular changes in the liver. Oil Red O and Masson's trichrome staining were used to evaluate lipid accumulation and fibrosis within the liver. The livers of MASLD mice exhibited increased lipid droplets and collagen fibers. Intriguingly, BATMen therapy alleviated these abnormalities in the livers of MASLD mice (FIG. 4a). Moreover, at the molecular level, the upregulation of fibrosis and inflammation markers, such as Collagen type III alpha 1 chain (Col3a1) and Tumor necrosis factor alpha (TNFα), observed in MASLD mice was reversed by BATMen therapy, bringing these markers down to levels comparable to those in mice fed a normal diet (FIG. 4b).

### Example 5

### BATMen Regulate Immune Profiles Within Liver TME of MASLD Mice

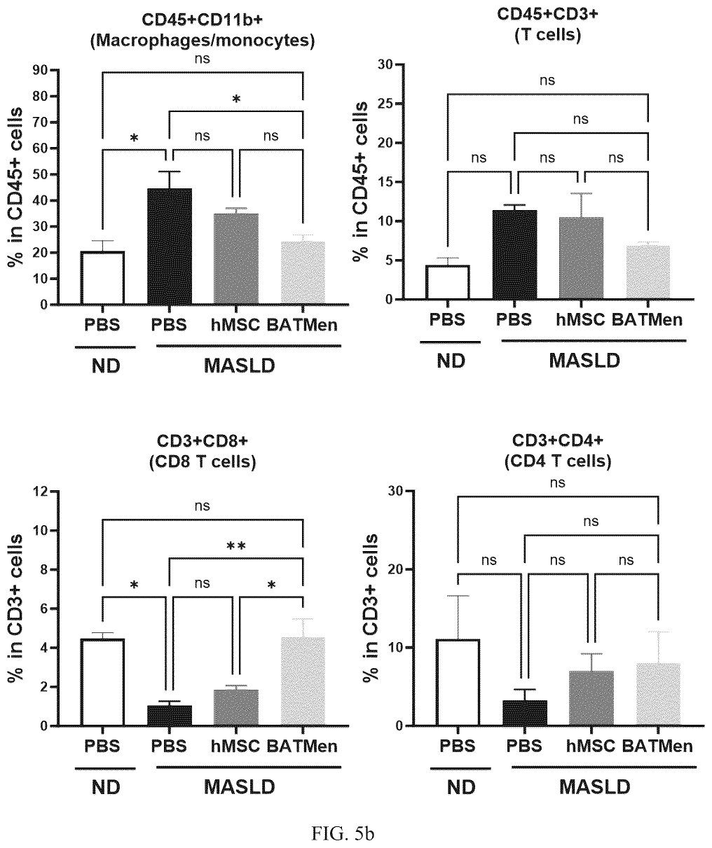

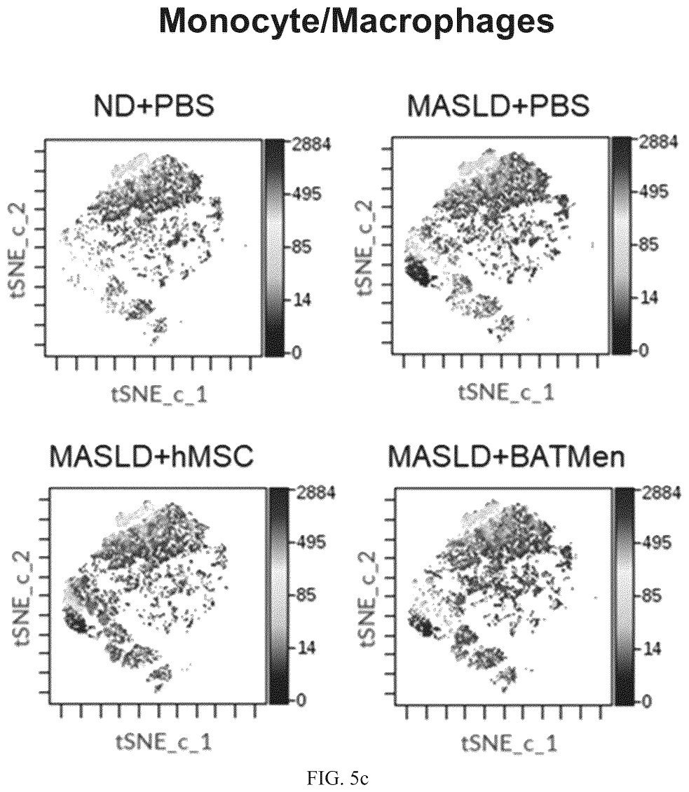

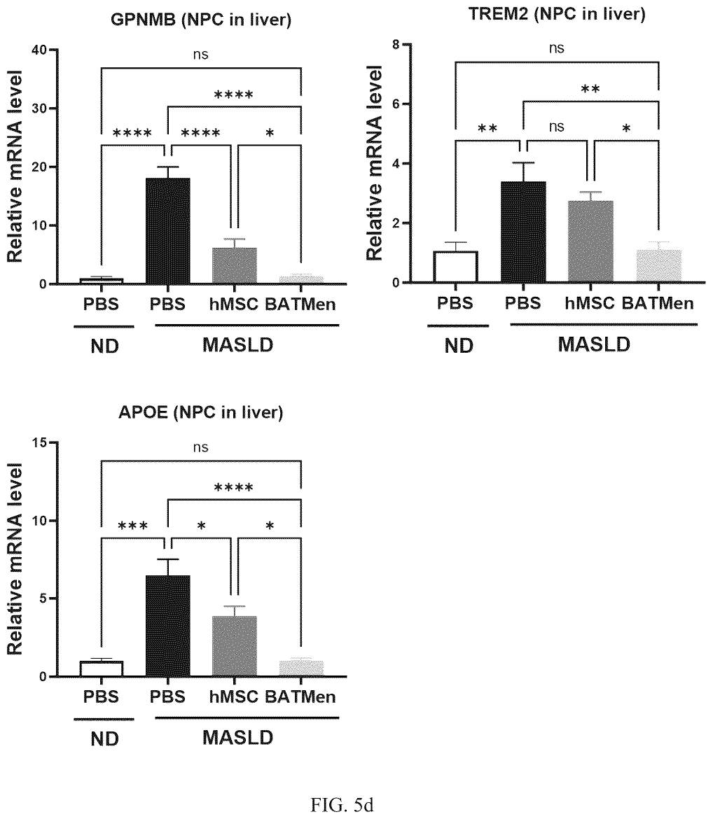

Chronic inflammation is a hallmark of MASLD development. It has been pointed out that the immunosuppressive tissue microenvironment (TME) of MASLD and HCC actually promotes cells evade host immune surveillance, leading to difficulty of therapy. Mouse BAT has the ability to resolve inflammation in TME of liver via secreted factor, To this end, we examined whether BATMen re-shapes immune regulation within TME of MASLD liver. After isolating non-parenchymal cell population (NPC) from liver, we applied those individual cells to CyTOF analysis to elaborate the immune profiles. The results revealed that MASLD liver was infiltrated more monocytes/macrophages and contained less CD8⁺ T cells (FIGS. 5a and 5b). Intriguingly, BATMen therapy reversed this immune profile within TME of MASLD liver. BATMen downregulated the population of monocytes/macrophages and upregulated CD8⁺ T cells (FIGS. 5a and 5b). Using tSEN analysis, the results showed that the specific subtype of monocytes/macrophages was dramatically increased in MASLD liver but it was decreased (left-bottom) after BATMen treatment (FIG. 5c). Following the assessment of MASLD-associated microphage (NAM) markers like GPNMB, TREM2, and APOE in non-parenchymal cells (NPC) isolated from mouse livers, we noted an increase in NAM markers in the liver following MASLD diet treatment (FIG. 5d). Notably, the administration of BATMen resulted in a decrease in the levels of NAM markers (FIG. 5d). These findings suggest that BATMen therapy effectively mitigates inflammation in the MASLD liver.

1. A population of genetically engineered mesenchymal stem cells (MSCs) comprising one, two or three exogenous genes selected from peroxisome proliferator-activated receptor gamma (PPARγ), peroxisome proliferator-activated receptor-γ coactivator 1-α (PGC1α) and mitochondrial uncoupling protein 1 (UCP1), wherein the gene expressions of PPARγ, PGC1α and UCP1 are increased.

2. The population of genetically engineered MSCs of claim 1, wherein the MSCs are umbilical cord mesenchymal stem cells (UMSCs), adipose derived mesenchymal stem cells (ADSCs), or bone marrow mesenchymal stem cells (BMSCs).

3. The population of genetically engineered MSCs of claim 1, wherein the exogenous genes are introduced to MSC by using a vector comprising PPARγ, PGC1α and UCP1 genes.

4. The population of genetically engineered MSCs of claim 3, wherein the vector is a transposon vector.

5. The population of genetically engineered MSCs of claim 1, wherein the exogenous genes are introduced to MSC by using clustered regularly interspaced short palindromic repeats (CRISPR)/nuclease.

6. The population of genetically engineered MSCs of claim 3, wherein the vector comprises dCas9-VP64 and MS2-p65-HSF1 fragments.

7. The population of genetically engineered MSCs of claim 3, wherein the vector comprises a U6 promoter operatively linked to the exogenous genes.

8. The population of genetically engineered MSCs of claim 3, wherein the vector is an all-in-one CRISPR-based multigene activation plasmid constructed by steps of:
providing gRNA cloning vectors comprising recombination sites and PPARγ, PGC1α and UCP1, respectively, and mixing the gRNA cloning vectors with a transposon backbone vector containing CRISPR/nuclease to generate all-in-one CRISPR-based multigene activation plasmid.

9. A vector comprising exogenous genes PPARγ, PGC1α and UCP1.

10. The vector of claim 9, which comprises dCas9-VP64 and MS2-p65-HSF1 fragments.

11. The vector of claim 9, which comprises a U6 promoter operatively linked to the exogenous genes.

12. A method for preparing the population of genetically engineered MSCs of claim 1, comprising preparing a vector comprising all-in-one CRISPR-based multigene activation plasmid, wherein the plasmid is prepared by providing gRNA cloning vectors comprising recombination sites and PPARγ, PGC1α and UCP1, respectively, and mixing the gRNA cloning vectors with a transposon backbone vector containing CRISPR/nuclease to generate all-in-one CRISPR-based multigene activation plasmid; transfecting MSCs with the vectors to generate genetically engineered MSCs, culturing the genetically engineered MSCs in a medium suitable for growth and propagation of the genetically engineered MSCs and harvesting a population of genetically engineered MSCs.

13. A method for preventing, ameliorating and/or treating a metabolic disorder in a subject in need thereof comprising administering a pharmaceutical composition to the subject, wherein the pharmaceutical composition comprising the population of genetically engineered MSCs of claim 1, and optionally a pharmaceutically acceptable carrier.

14. The method of claim 13, wherein the metabolic disorder is diabetes, obesity, central obesity, insulin-resistance, hypertension, metabolic dysfunction-associated steatotic liver disease (MASLD), an insulin resistance disorder, hepatic steatosis, cardiac deficiency, ischemic cardiac disease, high blood pressure, inflammation, triglyceride dyslipidemia, HDL dyslipidemia, cholesterol dyslipidemia, elevated fasting plasma glucose, leptin dysregulation, adipon dysregulation, or cancer.

15. The method of claim 13, wherein the method is for improving insulin sensitivity, promoting efficient energy utilization, reshaping immune cell populations, and/or improving lipid accumulation, inflammation or fibrosis within liver.

16. The method of claim 13, wherein the effective amount ranges from about 1×10⁴ cells to about 1×10⁸ cells.

17. A method for preventing, ameliorating and/or treating a metabolic disorder in a subject in need thereof, comprising administering a pharmaceutical composition to the subject, wherein the pharmaceutical composition comprising the population of genetically engineered MSCs of claim 8, and optionally a pharmaceutically acceptable carrier.

18. The method of claim 17, wherein the metabolic disorder is diabetes, obesity, central obesity, insulin-resistance, hypertension, metabolic dysfunction-associated steatotic liver disease (MASLD), an insulin resistance disorder, hepatic steatosis, cardiac deficiency, ischemic cardiac disease, high blood pressure, inflammation, triglyceride dyslipidemia, HDL dyslipidemia, cholesterol dyslipidemia, elevated fasting plasma glucose, leptin dysregulation, adipon dysregulation, or cancer.

19. The method of claim 18, wherein the method is for improving insulin sensitivity, promoting efficient energy utilization, reshaping immune cell populations, and/or improving lipid accumulation, inflammation or fibrosis within liver.

20. The method of claim 18, wherein the effective amount ranges from about 1×10⁴ cells to about 1×10⁸ cells.
